(19) United States
(12) Patent Application Publication
(10) Pub. No.: US 2023/0226544 A1
(43) Pub. Date: Jul. 20, 2023

(54) ION-SELECTIVE MEMBRANE, ION-SELECTIVE ELECTRODE, ION SENSOR, SPECIMEN TESTING DEVICE, AND COMPLEX COMPOUND

(71) Applicants: CANON KABUSHIKI KAISHA, Tokyo (JP); CANON MEDICAL SYSTEMS CORPORATION, Tochigi (JP)

(72) Inventors: WATARU KUBO, Tokyo (JP); HIROSHI SAITO, Kanagawa (JP); AKIO MARUYAMA, Tokyo (JP)

(21) Appl. No.: 18/145,351

(22) Filed: Dec. 22, 2022

(30) Foreign Application Priority Data
Jan. 20, 2022 (JP) 2022-007483
Dec. 13, 2022 (JP) 2022-198727

(51) Int. Cl. B01L 3/00; C08K 5/55; C08K 5/00; C08J 5/22; C07D 487/22

(52) U.S. Cl. CPC B01L 3/502715; C07D 487/22; C08J 5/22; C08K 5/55; C08K 5/0091; B01L 2300/0645; C08J 2327/06

(57) ABSTRACT

Provided is a high-stability ion-selective membrane (ISM) containing a thallium porphyrin complex as an ionophore, the ISM containing: a compound represented by the following formula (1); a polymer; and a membrane solvent.

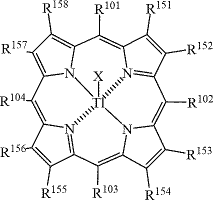

### BACKGROUND OF THE INVENTION

### Field of the Invention

The present invention relates to an ion-selective membrane, an ion-selective electrode, an ion sensor, a specimen testing device, and a complex compound.

### Description of the Related Art

An electrode that selectively performs a potential response to specific ions is called an ion-selective electrode (ISE). When the ISE is immersed in a solution, a potential difference corresponding to the activity of a target ion is generated at an interface between an ion-selective membrane and the solution to cause a change in electrode potential. An ion concentration measuring instrument (ion sensor) based on this phenomenon has been widely used in the fields of physics and chemistry industry, agriculture, medicine, food, environment, and the like. In a specimen testing device for medicine, an ion-selective electrode has been widely used in order to measure the concentrations of sodium, potassium, and chloride ions. For sodium and potassium ions that are cations among those ions, an ionophore-type ISE using an ion-selective membrane (ISM) in which a substance for selecting ions called an ionophore is confined in a hydrophobic membrane has been widely used. In contrast, for a chloride ion that is an anion, there is no ionophore as excellent as those for sodium and potassium ions, and a poorly-soluble salt type ISE such as a silver-silver chloride electrode and an ion-exchange type ISE using, for example, an ion-exchange resin having an ammonium salt or the like immobilized thereto are used in many cases.

Ionophores for a chloride ion are roughly classified into three kinds: an organometallic type, an organic type, and a metal complex type. Of those, the organometallic type exhibits high chloride ion selectivity, but in actuality, is difficult to use from the viewpoint of toxicity of chemical substances because of the use of organomercury and organotin compounds, and the like. In addition, the organic type such as bisthiourea has low chloride ion selectivity. The metal complex type exhibits high chloride ion selectivity, but is difficult to use from the viewpoint of the regulation of chemical substances because of the use of indium, manganese, and the like. In particular, regarding an indium compound, specific measures are required by, for example, the Ordinance on Prevention of Hazards Due to Specified Chemical Substances under the Industrial Safety and Health Law.

In Analytical Sciences, 1998, Vol. 14, pp. 79-84, there is a disclosure of a thallium porphyrin complex as an ionophore for a chloride ion. A thallium compound is not subject to the above-mentioned specific measures, and is preferred as compared to an indium complex from the viewpoint of the regulation of chemical substances. The inventors have made investigations on the characteristics of an existing thallium porphyrin complex and have recognized that the thallium porphyrin complex has high chloride ion selectivity. Meanwhile, the inventors have found that an ISM containing the existing thallium porphyrin complex is unstable and a change in electromotive force in response to an ion concentration is decreased with time, and that crystals of the thallium porphyrin complex are deposited on the surface of the ISM in this case. In view of the foregoing, the inventors have conceived that there is a problem in affinity of the existing thallium porphyrin complex for an ion-selective membrane matrix.

The present invention has been made to solve the above-mentioned problem, and an object of the present invention is to provide a high-stability ISM containing a thallium porphyrin complex as an ionophore.

### SUMMARY OF THE INVENTION

An ISM containing, as an ionophore, a thallium porphyrin complex (Tl-Por) having an appropriate substituent introduced thereto is provided.

That is, according to one embodiment of the present invention, there is provided an ion-selective membrane including: a compound represented by the following formula (1); a polymer; and a membrane solvent:

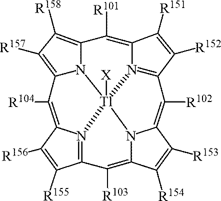

in the formula (1),
(i) R¹⁵¹ to R¹⁵⁸ and R¹⁰¹ to R¹⁰⁴ each independently represent a hydrogen atom, a saturated or unsaturated alkyl group having 1 or more and 20 or less carbon atoms that may have a substituent, an alkoxy group having 1 or more and 20 or less carbon atoms that may have a substituent, an alkylcarbonyl group having 1 or more and 20 or less carbon atoms that may have a substituent, an aryl group having 5 or more and 20 or less carbon atoms that may have a substituent, an aryloxy group having 5 or more and 20 or less carbon atoms that may have a substituent, an aralkyl group having 6 or more and 30 or less carbon atoms that may have a substituent, or a halogen atom,
(ii) the substituent of the alkyl group in the (i) is selected from the group consisting of: an alkoxy group having 1 or more and 4 or less carbon atoms; an alkylcarbonyl group having 1 or more and 4 or less carbon atoms; an alkyloxycarbonyl group having 1 or more and 4 or less carbon atoms; an alkylcarbonyloxy group having 1 or more and 4 or less carbon atoms; an aryl group having 5 or more and 20 or less carbon atoms; a cyano group; and a halogen atom,
(iii) the substituent of each of the alkoxy group, the alkylcarbonyl group, the aryl group, the aryloxy group, and the aralkyl group in the (i) is selected from the group consisting of: a saturated or unsaturated alkyl group having 1 or more and 20 or less carbon atoms; an alkoxy group having 1 or more and 20 or less carbon atoms; an alkylcarbonyl group having 1 or more and 20 or less carbon atoms; an alkyloxycarbonyl group having 1 or more and 20 or less carbon atoms; an alkylcarbonyloxy group having 1 or more and 20 or less carbon atoms; an aryl group having 5 or more and 20 or less carbon atoms; a cyano group; and a halogen atom,
(iv) at least one of the R¹⁵¹ to the R¹⁵⁸ and the R¹⁰¹ to the R¹⁰⁴ represents a group except a hydrogen atom, and
(v) X represents an anion.

Further features of the present invention will become apparent from the following description of exemplary embodiments with reference to the attached drawings.

### BRIEF DESCRIPTION OF THE DRAWINGS

FIG. 1 is a schematic view for illustrating an overview of an ISE.

FIG. 2 is a schematic view for illustrating an overview of an ISM.

FIG. 3 is a schematic view for illustrating an overview of an ion sensor using the ISE.

FIG. 4 is a schematic view for illustrating an overview of a specimen testing device.

FIG. 5A is a graph for showing results of Example 4 of an electromotive force response when the concentration of NaCl in an aqueous solution is changed. FIG. 5B is a graph for showing results of Comparative Example of an electromotive force response when the concentration of NaCl in an aqueous solution is changed.

### DESCRIPTION OF THE EMBODIMENTS

Embodiments of the present invention are described in detail below with reference to the drawings. However, the present invention is not limited to the following.

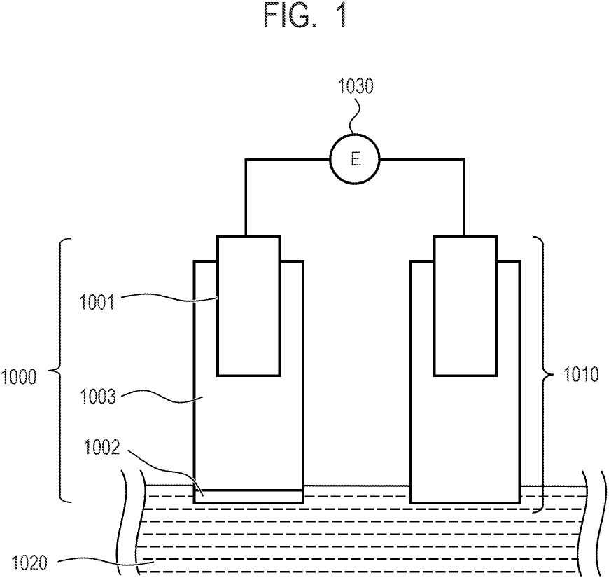

FIG. 1 is a schematic view for illustrating an overview of an ISE 1000 including an ISM 1002 of this embodiment. In FIG. 1, the ISE 1000 includes an electrode 1001 including a conductor and the ISM 1002. The ISE 1000 may contain an internal liquid 1003. The ISE 1000 is arranged in contact with a measurement solution 1020 together with a reference electrode 1010 serving as a comparison target. The reference electrode 1010 may be in contact with the measurement solution through a salt bridge or a liquid junction. In the ISM 1002 in contact with the measurement solution 1020, a membrane potential (E_{M}) proportional to the logarithm of the concentration (exactly, the activity a_{I}(aq)) of a target ion 2005 in the measurement solution 1020 is generated. The generated membrane potential may be represented by the following Nernst equation.

$${\text{E}}_{\text{M}} = {\text{E}}^{0} + \frac{\text{RT}}{\text{zF}} {\text{ln a}}_{\text{I}} \left(\text{aq}\right)$$

In the equation, E⁰ represents a standard electrode potential, R represents a gas constant, T represents an absolute temperature, “z” represents the number of charges of ions, and F represents a Faraday constant. In an ion sensor, the concentration of the target ion 2005 in the measurement solution 1020 is calculated by measuring a potential difference between the ISM 1002 and the reference electrode 1010 with a measuring instrument 1030 for each of a solution in which the concentration of the target ion 2005 is known and the measurement solution. Each of the constituent elements illustrated in FIG. 1 is specifically described below.

### Ion-Selective Electrode (ISE)

This embodiment provides an ion-selective electrode including: an electrode including at least one kind of conductor; and an ion-selective membrane of this embodiment.

Specifically, the ISE 1000 of this embodiment includes: the electrode 1001 including at least one kind of conductor; and the ISM 1002. The ISE 1000 may have a known structure. As an example, there is given such a structure as illustrated in FIG. 1 in which an internal electrode represented by the electrode 1001 including the conductor is in contact with the internal liquid 1003, the internal liquid 1003 is in contact with the ISM 1002, and the ISM 1002 is in contact with the measurement solution 1020. A known configuration may be used as the configuration of the ISE 1000 except the ISM 1002. Examples of a tubular body of the ISE 1000 include tubular bodies made of polymers, such as polyvinyl chloride and polymethyl methacrylate. Examples of the internal liquid 1003 include aqueous solutions of sodium chloride and potassium chloride. Examples of the internal electrode include: metal/poorly-soluble metal chloride such as silver-silver chloride; platinum; and conductive substances, such as a conductive carbon material and a conductive polymer. In addition, the fixed-type ISE 1000 in which the internal liquid 1003 is omitted, and the ISM 1002 is formed on the electrode 1001 including the conductor is also preferably used.

The ISE 1000 may be used, for example, in such a manner as illustrated in FIG. 1. Specifically, the ISE 1000 is arranged in contact with the measurement solution 1020 together with the reference electrode 1010. The reference electrode 1010 may be in contact with the measurement solution 1020 through a salt bridge or a liquid junction. A known reference electrode may be used as the reference electrode 1010. Specific examples thereof include a silver-silver chloride electrode, a conductive carbon material, a conductive polymer, and platinum.

### Ion-Selective Membrane (ISM)

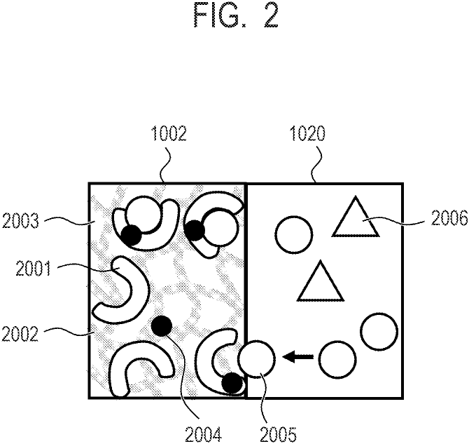

This embodiment provides an ion-selective membrane (ISM 1002) containing: a compound represented by the formula (1); a polymer; and a membrane solvent. An example of the ISM 1002 of this embodiment is described with reference to FIG. 2. The ISM 1002 of this embodiment contains an ionophore 2001, a polymer 2002, and a membrane solvent 2003. In addition, the ISM 1002 may contain an ionic additive 2004. The ionophore 2001 enables the generation of an ion-selective membrane potential by selectively incorporating the target ion 2005 into the ISM 1002 from the measurement solution 1020 containing the target ion 2005 and a foreign ion 2006 (process indicated by the arrow in the figure). A known method may be used as a method of preparing the ISM 1002 of this embodiment. An example thereof is described below. The ionophore 2001, the polymer 2002, the membrane solvent 2003, and the ionic additive 2004 are dissolved in a process solvent, and the solution is cast onto a glass plate or the like and left to stand. After the process solvent is volatilized, the resultant is formed into a desired shape. A solvent, which can dissolve the constituents of the ISM 1002 to a concentration that enables membrane formation, and which is suitable for membrane formation, is preferably used as the process solvent. Examples thereof include tetrahydrofuran, chloroform, acetone, methyl ethyl ketone, toluene, and ethyl acetate. The thickness of the ISM 1002 may be adjusted by controlling the amount of the process solvent and the like. A suitable thickness varies depending on mechanical strength, cost, and the like required in an assumed application, but values of 1 µm or more and 5 mm or less may be given as examples. The components for forming the ISM 1002 are described below.

### Membrane Solvent

The ISM of this embodiment contains the membrane solvent. The membrane solvent 2003 accounts for the largest mass ratio in the components for forming the ISM in many cases. The membrane solvent 2003 forms a flexible matrix together with the polymer 2002 and holds other constituent components such as an ionophore in the matrix, to thereby allow the ISM to function. The measurement solution is usually an aqueous solution, and hence the membrane solvent 2003 of the ISM has hydrophobicity that allows the membrane solvent 2003 to hold the constituent components of the ISM so that the constituent components may not be dissolved into the membrane. Meanwhile, it is preferred that the membrane solvent 2003 have such polarity as to be capable of satisfactorily holding the ionophore 2001 and the ionic additive 2004 each having polarity to some degree, and incorporating the target ion 2005 into the membrane. A known substance may be used as a substance of the membrane solvent 2003. Examples thereof include a phthalic acid ester, a fatty acid ester, an ortho-nitrophenyl ether, and a phosphoric acid ester. Further, a specific example thereof is at least one kind selected from dioctyl phthalate, dioctyl adipate, dioctyl sebacate, ortho-nitrophenyl octyl ether (NPOE), ortho-nitrophenyl phenyl ether, 2-fluoro-2′-nitrophenyl ether, and trioctyl phosphate. Of those, NPOE, dioctyl phthalate, and 2-fluoro-2′-nitrophenyl ether are preferably used. The composition ratio of the membrane solvent 2003 in the ISM may be appropriately selected depending on the application of the ion-sensitive membrane. As an example, there may be given a composition ratio of 10% or more and 90% or less of the mass of the ISM.

### Polymer

The ISM of this embodiment contains the polymer. The polymer 2002 has a mass ratio, which is the second largest after the membrane solvent 2003 in the components for forming the ISM, in many cases. The polymer 2002 forms the matrix together with the membrane solvent 2003 and functions to hold the shape of the ISM as a solid membrane. A polymer having low water solubility is preferably used as the polymer 2002 in order to form the ISM in contact with the measurement solution that is an aqueous solution and to form the matrix together with the hydrophobic membrane solvent 2003. A specific example of the polymer may be at least one kind selected from a polymer of ethylene that may have a substituent (e.g., a polymer of a vinyl halide or a polymer of vinyl acetate), a polymer of styrene that may have a substituent, a polymer of an acrylic acid ester, a polymer of a methacrylic acid ester, a polymer of a diene-based compound, polyurethane, a polymer having a siloxane bond, and a cellulose derivative. A further specific example thereof may be at least one kind selected from polyvinyl chloride, polystyrene, polymethyl acrylate, polymethyl methacrylate, polyvinyl acetate, polybutadiene, polyisoprene, polyacrylonitrile, and cellulose acetate.

### Ionophore

The ionophore in this embodiment is a compound that enables the generation of an ion-selective membrane potential by selectively incorporating the target ion 2005 from the measurement solution into the ISM. The ionophore 2001 in this embodiment is an ionophore having a structure of a thallium porphyrin complex represented by the formula (1). As ionophores for a chloride ion, three kinds are known: an organometallic type, an organic type, and a metal complex type. Of those, the organometallic type has toxicity, and the organic type has low ion selectivity. Thus, those types have problems in terms of actual use. An indium porphyrin complex that is known as the metal complex type has toxicity, albeit lower than that of the organometallic type, and is hence subject to the regulation. The inventors have changed a central metal of the existing indium porphyrin complex to thallium. That is, through use of a porphyrin complex in which thallium, which is one of the Group 13 elements that are large elements, is used as the central metal, the inventors have made the porphyrin complex less toxic and less subject to the regulation and the like as compared to the indium porphyrin complex.

The inventors have evaluated the characteristics of the thallium porphyrin complex in the initial investigations and have recognized that the thallium porphyrin complex exhibits high chloride ion selectivity. Meanwhile, the inventors have found that the ISM containing the thallium porphyrin complex has the following problems: the ISM is unstable and a change in electromotive force in response to an ion concentration is decreased with time; and crystals of the thallium porphyrin complex are deposited on the surface of the ISM in this case. In view of the foregoing, the inventors have conceived that the thallium porphyrin complex has a problem in affinity for an ion-selective membrane matrix. The thallium porphyrin complex is a π-conjugated spreading planar molecule, and hence may easily form an intermolecular association. That is, the inventors have conceived that, when the existing thallium porphyrin complex is used, the thallium porphyrin complex forms an association. Then, the inventors have conceived as described below. Such association has low solubility to the membrane solvent, and hence the thallium porphyrin complex is deposited from the ISM with time, with the result that the effective ionophore concentration is decreased, and the electromotive force is decreased with time. Then, in order to solve the above-mentioned problems, the inventors have performed molecular design of an ionophore with a high affinity for the matrix formed of the membrane solvent and the polymer. Specifically, the inventors have performed the molecular design from the following two viewpoints and have made verifications. That is, the two viewpoints are (1) improvement in affinity for the matrix through introduction of a substituent and (2) suppression of association by steric hindrance through introduction of the substituent.

The viewpoint (1) is a method involving introducing a relatively flexible functional group into a π-conjugated spreading porphyrin ring, to thereby improve the affinity for the membrane solvent and the polymer that are relatively flexible molecules. The viewpoint (2) is molecular design involving increasing the bulk of a Tl-Por in a direction perpendicular to the porphyrin ring by steric hindrance of an introduced substituent to increase the distance between Tl-Por molecules, to thereby suppress the formation of an association between the Tl-Por molecules. This effect is conceived to be exhibited in a substituent-introduced Tl-Por as a whole. First, a substituent is introduced into an unsubstituted Tl-Por to introduce the effect of steric hindrance, to thereby increase the distance between Tl-Por molecules, with the result that intermolecular association can be reduced. Second, regarding a Tl-Por having a phenyl group introduced into a meso-position of porphyrin, the plane of the introduced phenyl group has a function of steric hindrance in that the plane stands to some degree with respect to the plane of a porphyrin ring even when the group is unsubstituted, and hence such Tl-Por is more advantageous than the unsubstituted Tl-Por is. It is effective to introduce a substituent into the introduced phenyl group because the plane of the phenyl group is allowed to further stand with respect to the plane of the porphyrin ring. From this viewpoint, it is effective to introduce substituents into an ortho-position and a meta-position relative to the porphyrin bonding position of the phenyl group introduced into the meso-position of the porphyrin. When the formation of an association between Tl-Por molecules is suppressed, and the affinity of the Tl-Por for the matrix is improved, the Tl-Por can be stably held in the ISM. With this configuration, a change in effective ionophore concentration with time can be suppressed. In addition, as a result, a change in electromotive force with time caused by ion selection can be suppressed. Further, when the affinity of the Tl-Por for the matrix can be improved, the effective concentration of the Tl-Por that can be stably held in the ISM can be improved. The forgoing leads to the improvement of an ion-selective electromotive force that is a characteristic of the ionophore. Specifically, the electromotive force with respect to the target ion can be improved to improve the sensitivity of an ion sensor.

Specifically, in the embodiment of the present invention, the ion-selective membrane may contain the ionophore represented by the formula (1):

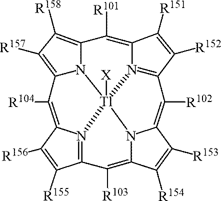

in the formula (1),
(i) R¹⁵¹ to R¹⁵⁸ and R¹⁰¹ to R¹⁰⁴ each independently represent a hydrogen atom, a saturated or unsaturated alkyl group having 1 or more and 20 or less carbon atoms that may have a substituent, an alkoxy group having 1 or more and 20 or less carbon atoms that may have a substituent, an alkylcarbonyl group having 1 or more and 20 or less carbon atoms that may have a substituent, an aryl group having 5 or more and 20 or less carbon atoms that may have a substituent, an aryloxy group having 5 or more and 20 or less carbon atoms that may have a substituent, an aralkyl group having 6 or more and 30 or less carbon atoms that may have a substituent, or a halogen atom,
(ii) the substituent of the alkyl group in the (i) is selected from the group consisting of: an alkoxy group having 1 or more and 4 or less carbon atoms; an alkylcarbonyl group having 1 or more and 4 or less carbon atoms; an alkyloxycarbonyl group having 1 or more and 4 or less carbon atoms; an alkylcarbonyloxy group having 1 or more and 4 or less carbon atoms; an aryl group having 5 or more and 20 or less carbon atoms; a cyano group; and a halogen atom,
(iii) the substituent of each of the alkoxy group, the alkylcarbonyl group, the aryl group, the aryloxy group, and the aralkyl group in the (i) is selected from the group consisting of: a saturated or unsaturated alkyl group having 1 or more and 20 or less carbon atoms; an alkoxy group having 1 or more and 20 or less carbon atoms; an alkylcarbonyl group having 1 or more and 20 or less carbon atoms; an alkyloxycarbonyl group having 1 or more and 20 or less carbon atoms; an alkylcarbonyloxy group having 1 or more and 20 or less carbon atoms; an aryl group having 5 or more and 20 or less carbon atoms; a cyano group; and a halogen atom,
(iv) at least one of the R¹⁵¹ to the R¹⁵⁸ and the R¹⁰¹ to the R¹⁰⁴ represents a group except a hydrogen atom, and
(v) X represents an anion.

In addition, in this embodiment, the ion-selective membrane may contain an ionophore represented by the formula (2):

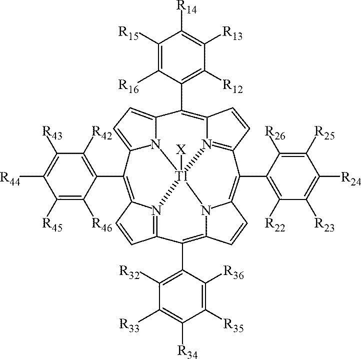

in the formula (2),
(i) R¹² to R¹⁶, R²² to R²⁶, R³² to R³⁶, and R⁴² to R⁴⁶ each independently represent a hydrogen atom, a saturated or unsaturated alkyl group having 1 or more and 20 or less carbon atoms that may have a substituent, an alkoxy group having 1 or more and 20 or less carbon atoms that may have a substituent, an alkylcarbonyl group having 1 or more and 20 or less carbon atoms that may have a substituent, an aryl group having 5 or more and 20 or less carbon atoms that may have a substituent, an aryloxy group having 5 or more and 20 or less carbon atoms that may have a substituent, an aralkyl group having 6 or more and 30 or less carbon atoms that may have a substituent, a hydroxy group, or a halogen atom,
(ii) the substituent of the alkyl group in the (i) is any one of an alkoxy group having 1 or more and 4 or less carbon atoms, an alkylcarbonyl group having 1 or more and 4 or less carbon atoms, an alkyloxycarbonyl group having 1 or more and 4 or less carbon atoms, an alkylcarbonyloxy group having 1 or more and 4 or less carbon atoms, an aryl group having 5 or more and 20 or less carbon atoms, a cyano group, or a halogen atom,
(iii) the substituent of each of the alkoxy group, the alkylcarbonyl group, the aryl group, the aryloxy group, and the aralkyl group in the (i) is any one of a saturated or unsaturated alkyl group having 1 or more and 4 or less carbon atoms, an alkoxy group having 1 or more and 4 or less carbon atoms, an alkylcarbonyl group having 1 or more and 4 or less carbon atoms, an alkyloxycarbonyl group having 1 or more and 4 or less carbon atoms, an alkylcarbonyloxy group having 1 or more and 4 or less carbon atoms, an aryl group having 5 or more and 20 or less carbon atoms, a cyano group, or a halogen atom,
(iv) at least one of the R¹² to the R¹⁶, the R²² to the R²⁶, the R³² to the R³⁶, and the R⁴² to the R⁴⁶ represents a group except a hydrogen atom, and
(v) X represents an anion.

In the formula (2), it is more preferred that the R¹² to the R¹⁶, the R²² to the R¹⁶, the R³² to the R³⁶, and the R⁴² to the R⁴⁶ each independently represent a hydrogen atom, a saturated or unsaturated alkyl group having 1 or more and 4 or less carbon atoms that may have a substituent, an alkoxy group having 1 or more and 4 or less carbon atoms that may have a substituent, an alkylcarbonyl group having 1 or more and 4 or less carbon atoms that may have a substituent, an aryl group having 5 or more and 10 or less carbon atoms that may have a substituent, an aryloxy group having 5 or more and 10 or less carbon atoms that may have a substituent, an aralkyl group having 6 or more and 10 or less carbon atoms that may have a substituent, a hydroxy group, or a halogen atom.

It is still more preferred that the R¹² to the R¹⁶, the R¹² to the R²⁶, the R³² to the R³⁶, and the R⁴² to the R⁴⁶ each independently represent a hydrogen atom, a saturated or unsaturated alkyl group having 1 or more and 4 or less carbon atoms that may have a substituent, or an alkoxy group having 1 or more and 4 or less carbon atoms that may have a substituent. It is particularly preferred that the R¹⁴, the R²⁴, the R³⁴, and the R⁴⁴ each represent a saturated or unsaturated alkyl group having 1 or more and 4 or less carbon atoms, the R¹⁴, the R²⁴, the R³⁴, and the R⁴⁴ each represent an alkoxy group having 1 or more and 4 or less carbon atoms, the R¹³, the R¹⁵, the R²³, the R²⁵, the R³³, the R³⁵, the R⁴³, and the R⁴⁵ each represent a saturated or unsaturated alkyl group having 1 or more and 4 or less carbon atoms, or the R¹³, the R¹⁵, the R²³, the R²⁵, the R³³, the R³⁵, the R⁴³, and the R⁴⁵ each represent an alkoxy group having 1 or more and 4 or less carbon atoms.

A case in which the R¹⁴, the R²⁴, the R³⁴, and the R⁴⁴ each represent an alkoxy group having 1 or more and 4 or less carbon atoms, or a case in which the R¹³, the R¹⁵, the R²³, the R²⁵, the R³³, the R³⁵, the R⁴³, and the R⁴⁵ each represent an alkoxy group having 1 or more and 4 or less carbon atoms is most preferred.

In addition, in this embodiment, the ion-selective membrane may contain an ionophore represented by the formula (3):

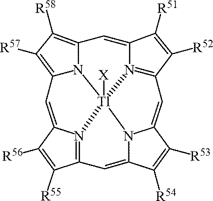

in the formula (3),
(i) R⁵¹ to R⁵⁸ each independently represent a hydrogen atom, a saturated or unsaturated alkyl group having 1 or more and 20 or less carbon atoms that may have a substituent, an alkoxy group having 1 or more and 20 or less carbon atoms that may have a substituent, an alkylcarbonyl group having 1 or more and 20 or less carbon atoms that may have a substituent, an aryl group having 5 or more and 20 or less carbon atoms that may have a substituent, an aryloxy group having 5 or more and 20 or less carbon atoms that may have a substituent, an aralkyl group having 6 or more and 30 or less carbon atoms that may have a substituent, or a halogen atom,
(ii) the substituent of the alkyl group in the (i) is any one of an alkoxy group having 1 or more and 4 or less carbon atoms, an alkylcarbonyl group having 1 or more and 4 or less carbon atoms, an alkyloxycarbonyl group having 1 or more and 4 or less carbon atoms, an alkylcarbonyloxy group having 1 or more and 4 or less carbon atoms, an aryl group having 5 or more and 20 or less carbon atoms, a cyano group, or a halogen atom,
(iii) the substituent of each of the alkoxy group, the alkylcarbonyl group, the aryl group, the aryloxy group, and the aralkyl group in the (i) is any one of a saturated or unsaturated alkyl group having 1 or more and 4 or less carbon atoms, an alkoxy group having 1 or more and 4 or less carbon atoms, an alkylcarbonyl group having 1 or more and 4 or less carbon atoms, an alkyloxycarbonyl group having 1 or more and 4 or less carbon atoms, an alkylcarbonyloxy group having 1 or more and 4 or less carbon atoms, an aryl group having 5 or more and 20 or less carbon atoms, a cyano group, or a halogen atom,
(iv) at least one of the R⁵¹ to the R⁵⁸ represents a group except a hydrogen atom, and
(v) X represents an anion.

In the formula (3), it is more preferred that the R⁵¹ to the R⁵⁸ each independently represent a hydrogen atom, a saturated or unsaturated alkyl group having 1 or more and 4 or less carbon atoms that may have a substituent, an alkoxy group having 1 or more and 4 or less carbon atoms that may have a substituent, an alkylcarbonyl group having 1 or more and 4 or less carbon atoms that may have a substituent, an aryl group having 5 or more and 10 or less carbon atoms that may have a substituent, an aryloxy group having 5 or more and 10 or less carbon atoms that may have a substituent, an aralkyl group having 6 or more and 10 or less carbon atoms that may have a substituent, or a halogen atom. It is particularly preferred that the R⁵¹ to the R⁵⁸ each independently represent a hydrogen atom, or a saturated or unsaturated alkyl group having 1 or more and 4 or less carbon atoms that may have a substituent, and a particularly preferred example of the substituent is an alkyloxycarbonyl group having 1 or more and 4 or less carbon atoms.

In addition, in the formula (3), it is more preferred that the R⁵¹ to R⁵⁸ each have a group except a hydrogen atom. Such ionophore is further increased in bulk in a direction perpendicular to the plane of the porphyrin ring, and can more effectively suppress the formation of an ionophore association.

In addition, in this embodiment, the ion-selective membrane may contain an ionophore represented by the formula (4):

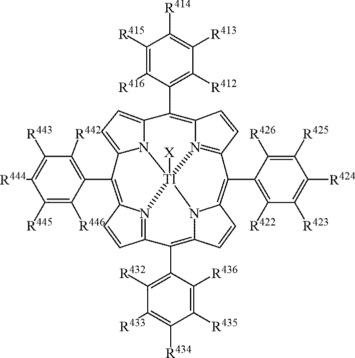

in the formula (4),
(i) R⁴¹² to R⁴¹⁶, R⁴²² to R⁴²⁶, R⁴³² to R⁴³⁶, and R⁴⁴² to R⁴⁴⁶ each independently represent a hydrogen atom, a saturated or unsaturated alkyl group having 1 or more and 20 or less carbon atoms that may have a substituent, an alkoxy group having 1 or more and 20 or less carbon atoms that may have a substituent, an alkylcarbonyl group having 1 or more and 20 or less carbon atoms that may have a substituent, an aryl group having 5 or more and 20 or less carbon atoms that may have a substituent, an aryloxy group having 5 or more and 20 or less carbon atoms that may have a substituent, an aralkyl group having 6 or more and 30 or less carbon atoms that may have a substituent, a hydroxy group, or a halogen atom,
(ii) the substituent of the alkyl group in the (i) is any one of an alkoxy group having 1 or more and 4 or less carbon atoms, an alkylcarbonyl group having 1 or more and 4 or less carbon atoms, an alkyloxycarbonyl group having 1 or more and 4 or less carbon atoms, an alkylcarbonyloxy group having 1 or more and 4 or less carbon atoms, an aryl group having 5 or more and 20 or less carbon atoms, a cyano group, or a halogen atom,
(iii) the substituent of each of the alkoxy group, the alkylcarbonyl group, the aryl group, the aryloxy group, and the aralkyl group in the (i) is any one of a saturated or unsaturated alkyl group having 1 or more and 4 or less carbon atoms, an alkoxy group having 1 or more and 4 or less carbon atoms, an alkylcarbonyl group having 1 or more and 4 or less carbon atoms, an alkyloxycarbonyl group having 1 or more and 4 or less carbon atoms, an alkylcarbonyloxy group having 1 or more and 4 or less carbon atoms, an aryl group having 5 or more and 20 or less carbon atoms, a cyano group, or a halogen atom,
(iv) at least one of the R⁴¹² to the R⁴¹⁶, the R⁴²² to the R⁴²⁶, the R⁴³² to the R⁴³⁶, and the R⁴⁴² to the R⁴⁴⁶ represents a group except a hydrogen atom,
(v) X represents an anion, and
(vi) at least one of the R⁴¹³, the R⁴¹⁵, the R⁴²³, the R⁴²⁵, the R⁴³³, the R⁴³⁵, the R⁴⁴³, or the R⁴⁴⁵ represents an alkoxy group having 1 or more and 20 or less carbon atoms that may have a substituent.

In such ionophore, —O— of the alkoxy group has hydrophilicity, and has flexibility. Accordingly, when such ionophore is incorporated into the ISM, the deterioration of the ionophore with time can be further reduced.

In the formula (4), it is more preferred that the R⁴¹² to the R⁴¹⁶, the R⁴²² to the R⁴²⁶, the R⁴³² to the R⁴³⁶, and the R⁴⁴² to the R⁴⁴⁶ each independently represent a hydrogen atom, a saturated or unsaturated alkyl group having 1 or more and 4 or less carbon atoms that may have a substituent, an alkoxy group having 1 or more and 4 or less carbon atoms that may have a substituent, an alkylcarbonyl group having 1 or more and 4 or less carbon atoms that may have a substituent, an aryl group having 5 or more and 10 or less carbon atoms that may have a substituent, an aryloxy group having 5 or more and 10 or less carbon atoms that may have a substituent, an aralkyl group having 5 or more and 10 or less carbon atoms that may have a substituent, a hydroxy group, or a halogen atom.

It is preferred that the R⁴¹³, the R⁴¹⁵, the R⁴²³, the R⁴²⁵, the R⁴³³, the R⁴³⁵, the R⁴⁴³, and the R⁴⁴⁵ each represent an alkoxy group having 1 or more and 20 or less carbon atoms that may have a substituent. It is more preferred that the R⁴¹³, the R⁴¹⁵, the R⁴²³, the R⁴²⁵, the R⁴³³, the R⁴³⁵, the R⁴⁴³, and the R⁴⁴⁵ each represent an alkoxy group having 1 or more and 4 or less carbon atoms that may have a substituent.

In such ionophore, —O— of the alkoxy group has hydrophilicity, and has flexibility. Accordingly, when such ionophore is incorporated into the ISM, the deterioration of the ionophore with time can be further reduced. Further, such ionophore has 8 alkoxy groups, and hence as compared to a case in which the number of alkoxy groups is small, the ionophore is further increased in bulk in a direction perpendicular to the plane of the porphyrin ring, and can more effectively suppress the formation of an ionophore association.

In addition, another embodiment provides a complex compound represented by the formula (4):

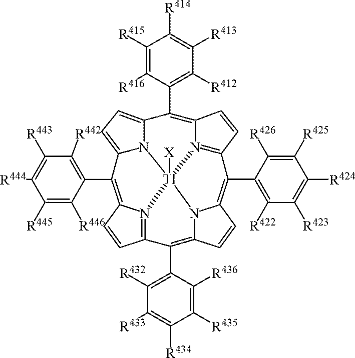

in the formula (4),
(i) R⁴¹² to R⁴¹⁶, R⁴²² to R⁴²⁶, R⁴³² to R⁴³⁶, and R⁴⁴² to R⁴⁴⁶ each independently represent a hydrogen atom, a saturated or unsaturated alkyl group having 1 or more and 20 or less carbon atoms that may have a substituent, an alkoxy group having 1 or more and 20 or less carbon atoms that may have a substituent, an alkylcarbonyl group having 1 or more and 20 or less carbon atoms that may have a substituent, an aryl group having 5 or more and 20 or less carbon atoms that may have a substituent, an aryloxy group having 5 or more and 20 or less carbon atoms that may have a substituent, an aralkyl group having 6 or more and 30 or less carbon atoms that may have a substituent, a hydroxy group, or a halogen atom,
(ii) the substituent of the alkyl group in the (i) is any one of an alkoxy group having 1 or more and 4 or less carbon atoms, an alkylcarbonyl group having 1 or more and 4 or less carbon atoms, an alkyloxycarbonyl group having 1 or more and 4 or less carbon atoms, an alkylcarbonyloxy group having 1 or more and 4 or less carbon atoms, an aryl group having 5 or more and 20 or less carbon atoms, a cyano group, or a halogen atom,
(iii) the substituent of each of the alkoxy group, the alkylcarbonyl group, the aryl group, the aryloxy group, and the aralkyl group in the (i) is any one of a saturated or unsaturated alkyl group having 1 or more and 4 or less carbon atoms, an alkoxy group having 1 or more and 4 or less carbon atoms, an alkylcarbonyl group having 1 or more and 4 or less carbon atoms, an alkyloxycarbonyl group having 1 or more and 4 or less carbon atoms, an alkylcarbonyloxy group having 1 or more and 4 or less carbon atoms, an aryl group having 5 or more and 20 or less carbon atoms, a cyano group, or a halogen atom,
(iv) at least one of the R⁴¹² to the R⁴¹⁶, the R⁴²² to the R⁴²⁶, the R⁴³² to the R⁴³⁶, and the R⁴⁴² to the R⁴⁴⁶ represents a group except a hydrogen atom,
(v) X represents an anion, and
(vi) at least one of the R⁴¹³, the R⁴¹⁵, the R⁴²³, the R⁴²⁵, the R⁴³³, the R⁴³⁵, the R⁴⁴³, or the R⁴⁴⁵ represents an alkoxy group having 1 or more and 20 or less carbon atoms that may have a substituent.

In the formula (4), it is more preferred that the R⁴¹² to the R⁴¹⁶, the R⁴²² to the R⁴²⁶, the R⁴³² to the R⁴³⁶, and the R⁴⁴² to the R⁴⁴⁶ each independently represent a hydrogen atom, a saturated or unsaturated alkyl group having 1 or more and 4 or less carbon atoms that may have a substituent, an alkoxy group having 1 or more and 4 or less carbon atoms that may have a substituent, an alkylcarbonyl group having 1 or more and 4 or less carbon atoms that may have a substituent, an aryl group having 5 or more and 10 or less carbon atoms that may have a substituent, an aryloxy group having 5 or more and 10 or less carbon atoms that may have a substituent, an aralkyl group having 5 or more and 10 or less carbon atoms that may have a substituent, a hydroxy group, or a halogen atom.

It is preferred that the R⁴¹³, the R⁴¹⁵, the R⁴²³, the R⁴²⁵, the R⁴³³, the R⁴³⁵, the R⁴⁴³, and the R⁴⁴⁵ each represent an alkoxy group having 1 or more and 20 or less carbon atoms that may have a substituent. It is more preferred that the R⁴¹³, the R⁴¹⁵, the R⁴²³, the R⁴²⁵, the R⁴³³, the R⁴³⁵, the R⁴⁴³, and the R⁴⁴⁵ each represent an alkoxy group having 1 or more and 4 or less carbon atoms that may have a substituent.

In addition, another embodiment provides a complex compound represented by the formula (5):

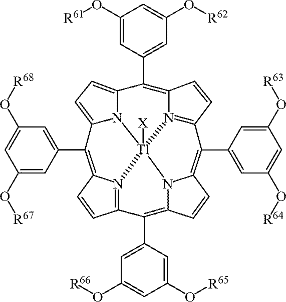

in the formula (5),
R⁶¹ to R⁶⁸ each independently represent a saturated or unsaturated alkyl group having 1 or more and 20 or less carbon atoms that may have a substituent,
the substituent is selected from the group consisting of: an alkoxy group having 1 or more and 4 or less carbon atoms; an alkylcarbonyl group having 1 or more and 4 or less carbon atoms; an alkyloxycarbonyl group having 1 or more and 4 or less carbon atoms; an alkylcarbonyloxy group having 1 or more and 4 or less carbon atoms; an aryl group having 5 or more and 20 or less carbon atoms; a cyano group; and a halogen atom, and
X represents an anion.

In the formula (5), it is more preferred that the R⁶¹ to the R⁶⁸ each independently represent a saturated alkyl group having 1 or more and 4 or less carbon atoms that may have a substituent, and as the most preferred example, there may be given a compound in which the R⁶¹ to the R⁶⁸ each represent a methyl group.

The trivalent valence of thallium that is a central metal of the ionophore exhibits a high degree of chloride ion selectivity. In order to achieve this, the anion X in each of the formulae (1) to (5) is preferably a stable anion, and the anion X is preferably a halide ion or an atomic group for forming a stable anion. The valence of thallium is held at a trivalent valence by selecting a halide ion or an atomic group for forming a stable anion. A known anion may be used as the anion X. An example of the anion X is any one of a chloride ion, a bromide ion, an iodide ion, a fluoride ion, a trifluoroacetate ion, a tetrafluoroborate ion, a hexafluorophosphate ion, an acetate ion, a nitrate ion, a perchlorate ion, or a thiocyanate ion. In particular, a chloride ion is most preferably used as the anion X because the chloride ion exhibits satisfactory chloride ion selectivity.

Specific structural formulae of the Tl-Por that is the ionophore according to this embodiment are illustrated below. However, the ionophore according to this embodiment is not limited thereto.

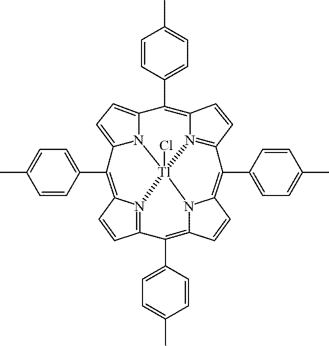

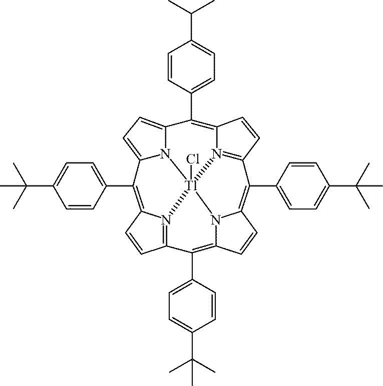

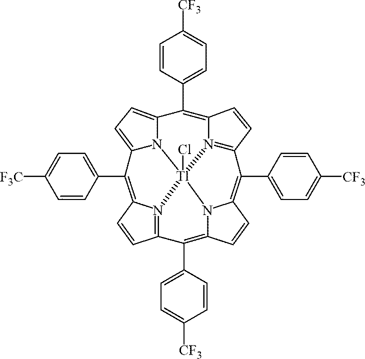

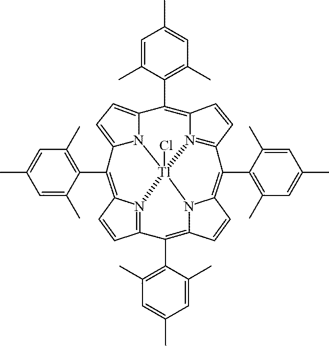

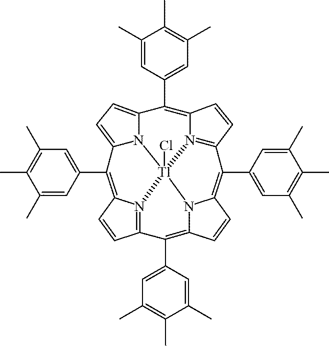

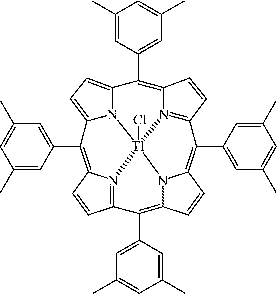

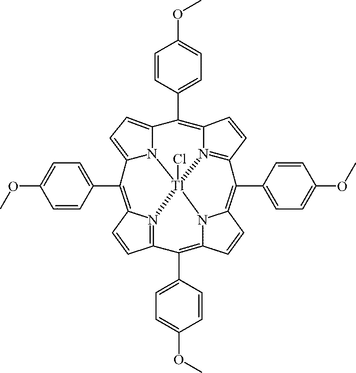

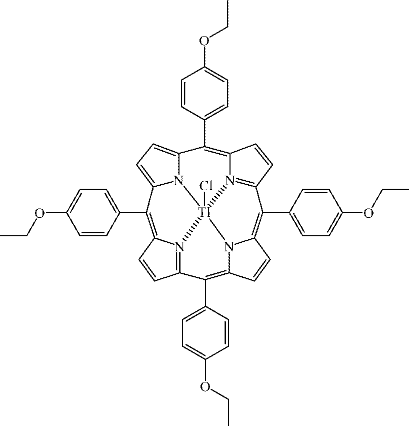

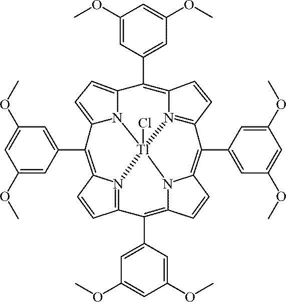

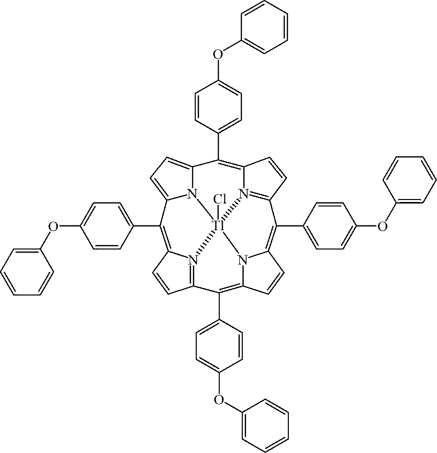

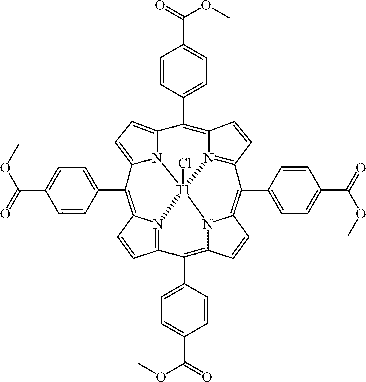

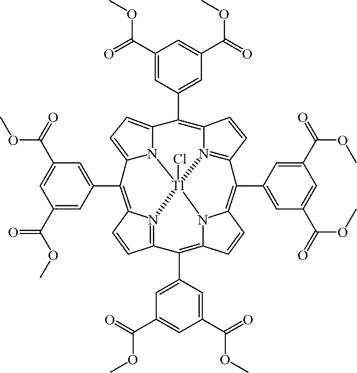

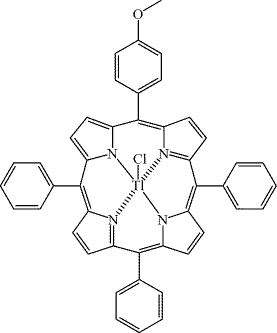

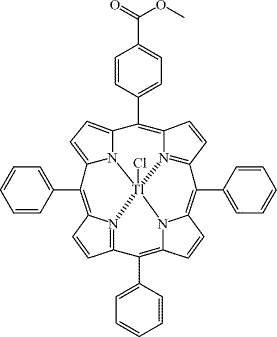

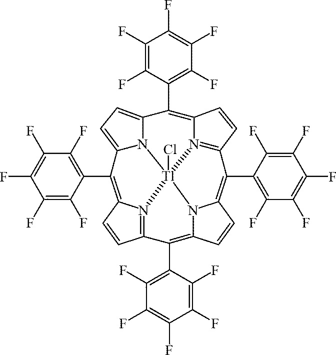

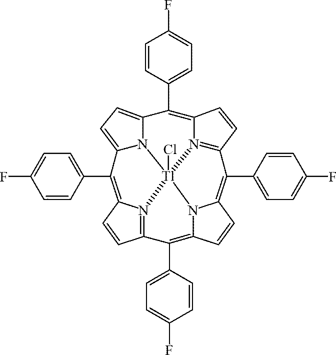

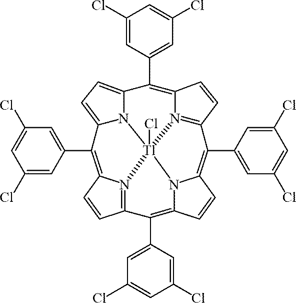

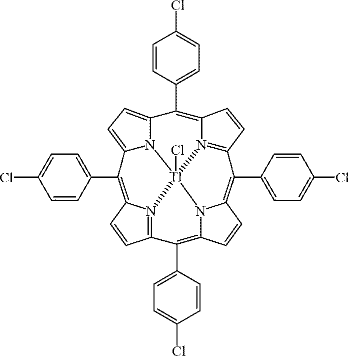

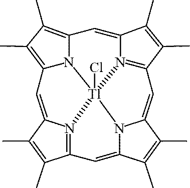

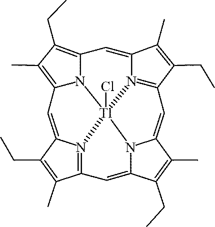

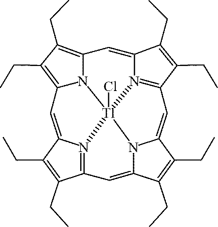

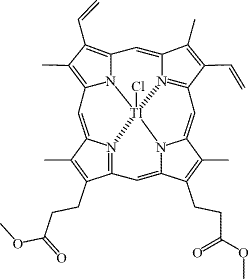

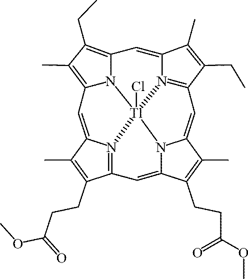

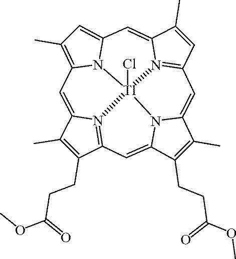

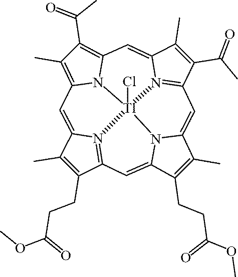

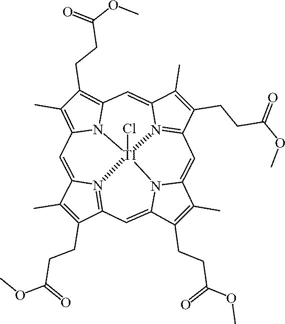

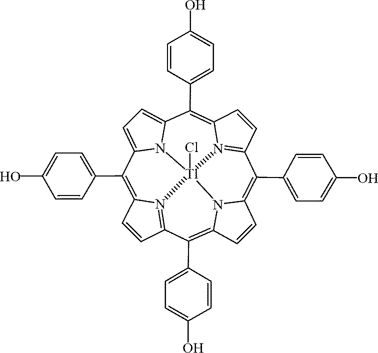

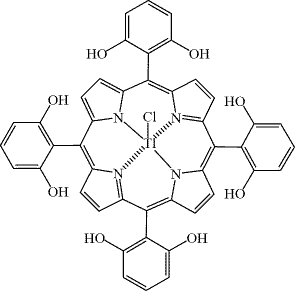

The amount of the Tl-Por incorporated into the ISM of this embodiment may be appropriately selected depending on the application of the ion-selective membrane. The content of the Tl-Por may be set to 0.1 mass% or more and 20 mass% or less of the amount of the ISM. The content may be set to preferably 0.5 mass% or more and 10 mass% or less. When the amount of the Tl-Por incorporated into the ISM is less than 0.1 mass%, the electromotive force response of the ISM to the target ion may be decreased. Meanwhile, when the amount of the Tl-Por incorporated into the ISM is more than 20 mass%, the Tl-Por may be deposited to inhibit the incorporation of the target ion into the membrane.

The ISM of this embodiment exhibits high selectivity for halide ions, in particular, a chloride ion through use of the Tl-Por. Specifically, the ISM exhibits high selectivity for a hydrogen carbonate ion, a phosphate ion, a nitrate ion, and the like, which are interfering ions present in biological fluids, such as blood and urine. Thus, through use of the ISM of this embodiment, an ISE with high selectivity for halide ions typified by a chloride ion can be formed.

### Ionic Additive

The ISM of this embodiment may contain the ionic additive. The ionic additive is said to have an action of preventing an ion that interferes with the detection of the target ion from entering the membrane. An ionic additive containing a hydrophilic cation and a hydrophobic anion is preferably used for the Tl-Por of this embodiment. A known anion may be used as the hydrophobic anion. As a method of recognizing hydrophobicity, there is given a method of recognizing whether or not a counter cation is insoluble in water when a tetrabutylammonium ion is used as the counter cation. Examples of the hydrophobic anion may include anions of the following compounds. That is, examples thereof include the anions of a tetraphenylborate compound, a long-chain alkyl sulfonic acid compound, a long-chain dialkylsulfosuccinic acid compound, a long-chain alkylphosphoric acid compound, a long-chain dialkylphosphoric acid compound, and a long-chain dialkylphosphosuccinic acid compound. Specific examples thereof include the anions of tetrakis[3,5-bis(trifluoromethyl)phenyl]borate (TFPB), tetrakis(4-chlorophenyl)borate, tetrakis(4-fluorophenyl)borate, tetraphenylborate, decylsulfonic acid, dodecylsulfonic acid, dodecylbenzenesulfonic acid, octadecylsulfonic acid, oleylsulfonic acid, bismethylhexylsulfosuccinic acid, dioctylsulfosuccinic acid, didecylsulfosuccinic acid, didodecylsulfosuccinic acid, decylphosphonic acid, dodecylphosphonic acid, dodecyl benzenephosphonic acid, octadecylphosphonic acid, oleylphosphonic acid, bismethylhexylphosphonic acid, dioctylphosphonic acid, didecylphosphonic acid, didodecylphosphonic acid, bismethylhexylphosphosuccinic acid, dioctylphosphosuccinic acid, didecylphosphosuccinic acid, and didodecylphosphosuccinic acid.

A hydrophilic cation is used as the cation of the ionic additive. As a method of recognizing hydrophilicity, there is given a method of recognizing whether or not a counter cation is soluble in water when a hexafluorophosphate ion is used as the counter cation. A known cation may be used as the cation of the ionic additive. Specifically, an alkali metal ion, an alkaline earth metal ion, an ammonium ion, or the like may be used as the cation of the ionic additive. Specific examples thereof include a sodium ion, a potassium ion, and an ammonium ion.

The content of the ionic additive in the ISM of this embodiment may be appropriately selected depending on the application of the ion-selective membrane. For example, the content of the ionic additive falls within a range of from 1 mol% to 100 mol% with respect to the Tl-Por. When the addition amount of the ionic additive is less than 1 mol% with respect to the Tl-Por, the electromotive force response of the ISM to the target ion may become slower. Meanwhile, when the addition amount of the ionic additive is more than 100 mol% with respect to the Tl-Por, the ISM exhibits a function as a cation exchanger more than a function of performing an electromotive force response to an anion, and hence may perform an electromotive force response to a cation. A more preferred concentration is, for example, as follows: the addition amount of the ionic additive falls within a range of from 10 mol% to 50 mol% with respect to the Tl-Por. In a general anion-selective electrode (e.g., bisthiourea), a combination of a hydrophobic cation and a hydrophilic anion is selected as its ionic additive in many cases. The Tl-Por uses a combination of the hydrophilic cation and the hydrophobic anion, and this is probably because its ion selectivity as the ionophore is activated by an interaction between the hydrophobic anion and the Tl-Por.

### Ion Sensor

This embodiment provides an ion sensor including: the ion-selective electrode of this embodiment; a reference electrode; and a measuring instrument configured to measure a potential difference between the ion-selective electrode and the reference electrode.

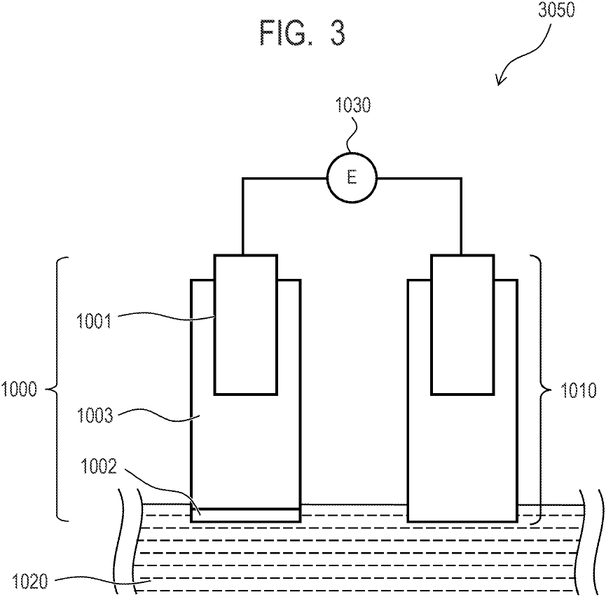

The ion sensor is a sensor that measures the concentration of a target ion in a sample. FIG. 3 is a schematic view for illustrating an overview of an ion sensor 3050 (ion concentration measuring device) using an ISE having the ISM of this embodiment formed thereon. In FIG. 3, the ISE 1000 is arranged in contact with the measurement solution 1020 together with the reference electrode 1010 serving as a comparison target. The reference electrode 1010 may be in contact with the measurement solution through a salt bridge or a liquid junction. In the ISM 1002 in contact with the measurement solution, a membrane potential (E_{M}) proportional to the logarithm of the concentration (exactly, the activity a_{I}(aq)) of the target ion in the measurement solution is generated. In the ion sensor (following the Nernst equation), the concentration of the target ion in the measurement solution is calculated by measuring a potential difference between the ISM 1002 and the reference electrode 1010 with the measuring instrument 1030 such as a voltmeter for each of a solution in which the concentration of the target ion is known and the measurement solution. The above-mentioned electrode is preferably used as the reference electrode 1010. A general measuring instrument may be used as the measuring instrument. For example, a voltmeter with a large input resistance or a potentiometer mode of a potentiostat is used. The input resistance is preferably 10⁹ Ω or more, more preferably 10¹² Ω or more.

### Specimen Testing Device

This embodiment provides a specimen testing device including: the ion-selective electrode of this embodiment; and a specimen supply mechanism configured to supply a specimen to the ion-selective electrode.

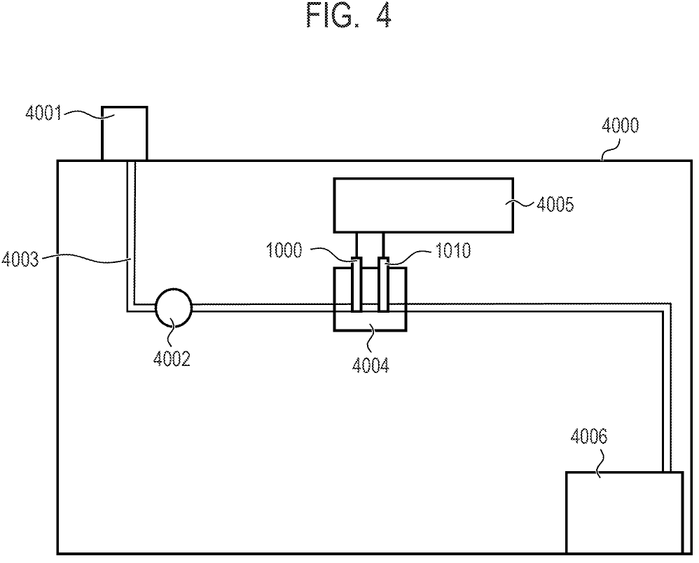

An example of the specimen testing device of this embodiment is described with reference to FIG. 4. A specimen testing device 4000 incorporates a specimen into the device from a specimen intake port 4001, and the specimen is supplied to the ISE 1000 and the reference electrode 1010 in an ISE unit 4004 through a flow path 4003 or the like with a power of a pump 4002 or the like. The electromotive force generated in the ISE 1000 is measured and recorded by a measuring unit 4005. The specimen sample used for the measurement is collected in a collection container 4006. In addition to the foregoing, the specimen testing device may include a unit that supplies and mixes a solution for diluting the specimen sample or a unit that measures any other test item. Through use of the ISE 1000 using the Tl-Por as an ionophore, the specimen testing device can analyze anions typified by a chloride ion with high accuracy.

The ISM of this embodiment can stably hold the Tl-Por in the ISM by improving the affinity for its matrix through use of the Tl-Por having a substituent as the ionophore. As a result, a change in effective ionophore concentration with time can be suppressed, and a change in electromotive force with time resulting from ion selection can be suppressed. When the effective concentration of the Tl-Por that can be stably held in the ISM is improved, the electromotive force resulting from the ion selection by the ionophore of the ISE can be improved. As a result, an ISE having improved electromotive force for the target ion, that is, an ion sensor with high sensitivity can be achieved. An ion sensor and a specimen testing device each using the ISE with high sensitivity and stability can measure the concentration of the target ion and perform a test on a specimen with high accuracy.

### EXAMPLES

This embodiment is hereinafter described more specifically by way of Examples, but the present invention is not limited to these Examples.

### Synthesis of Tl-Por

As each of compounds of Examples, a Tl-Por represented by each of the formulae P-1, P-4, P-7, P-9, P-21, P-22, and P-27 of exemplary compounds was synthesized by a reaction represented by the following formula (A) with reference to Non Patent Literature (Polyhedron1986, Vol. 5, pp. 1157-1164). In the formula (A), the synthetic reaction of the Tl-Por represented by the formula P-9 is described as a representative.

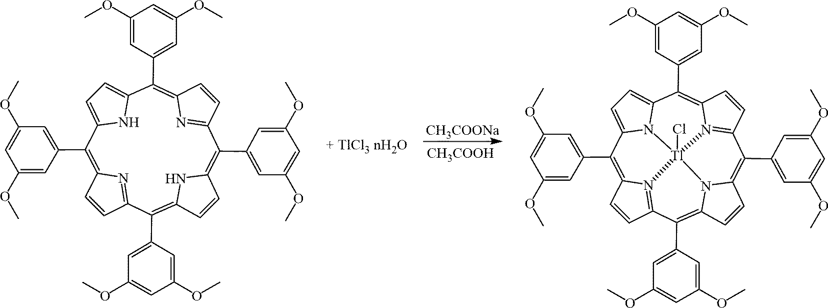

A more specific synthesis method is described by exemplifying the Tl-Por represented by the formula P-9 as a representative. Commercially available 5,10,15,20-tetrakis(3,5-dimethoxyphenyl)porphyrin and 2 equivalents of thallium(III) chloride were dissolved in acetic acid. Sodium acetate was added to the solution, and the mixture was refluxed at a power output of 100 W for 6 hours in a microwave irradiation device. The precipitate was collected by filtration and dried. The dried collected product was dissolved in toluene and filtered. The solvent was distilled off from the filtrate. Thus, the Tl-Por represented by the formula P-9 was obtained. The measurement results of ¹H-NMR of the Tl-Por represented by the formula P-9 were as described below.

¹H-NMR (deuterated acetone) δ (ppm): 9.24 (d, 8H), 7.56 (s, 4H), 7.40 (s, 4H), 7.02 (s, 4H), 4.00 (s, 24H)

A compound represented by the following formula R-1 of Comparative Example was also synthesized by the above-mentioned method.

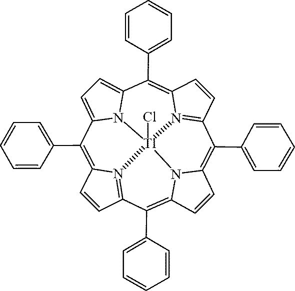

### Preparation of ISM

A method of preparing an ISM used in each of Examples and Comparative Example is described. The following materials were used. 2-Nitrophenyloctyl ether (NPOE) serving as a membrane solvent, polyvinyl chloride (PVC) serving as a polymer, a Tl-Por serving as an ionophore, sodium tetrakis[3,5-bis(trifluoromethyl)phenyl]borate (NaTFPB) serving as an ionic additive, and THF serving as a process solvent were used. NPOE, PVC, the Tl-Por, and NaTFPB were dissolved by heating in THF, and the resultant was cast onto a glass plate, followed by the volatilization of THF. Thus, the ISM was prepared. The mass ratio in the membrane was set to NPOE/PVC/Tl-Por=66/33/1. The concentration of the ionic additive was set to from 5.6 mol% to 100 mol% with respect to the Tl-Por. The thickness of the prepared ISM was about 0.4 mm.

### Production of ISE

A method of preparing an ISE used in each of Examples and Comparative Example is described. The ISM was cut out so as to fit an electrode size and welded to an electrode housing made of PVC with THF. The ISM was conditioned overnight with a 10 mM NaCl aqueous solution and then placed between a Ag/AgCl electrode and a sample liquid through an internal liquid formed of a 10 mM NaCl aqueous solution as illustrated in FIG. 1.

### Evaluation

### Evaluation of Solubility

The absorbance of the ISM was measured by changing the concentration of the Tl-Por to be dissolved in NPOE. The maximum concentration in a region in which the absorbance was increased in proportion to the concentration was defined as a saturation solubility in accordance with the Lambert-Beer law.

### Evaluation of Uniformity of ISM

The produced ISM was placed on a planar light source and a picture thereof was taken. The presence of large aggregates on the membrane surface and in the membrane was observed.

### Evaluation of Electromotive Force

The ISE and Ag/AgCl that was a reference electrode were connected to a potentiostat with an input resistance of 10¹² S2 that was a measuring instrument as illustrated in FIG. 1, and the concentration of NaCl in the aqueous solution was changed. An electromotive force (EMF) at this time was measured. The measurement was performed at 23° C.

### Evaluation of Stability

The NaCl concentration-dependent EMFs of the ISM were measured before and after the ISM was immersed in a 10 mM NaCl aqueous solution and was left to stand for 4 days, and the stability thereof was evaluated through use of a ratio of the slope of the EMF to the logarithm of the concentration (after standing/before standing).

### Results

### Evaluation of Solubility

The solubility of the Tl-Por represented by each of the formulae P-1, P-4, P-7, P-9, P-21, P-22, and P-27, which was a substituent-introduced Tl-Por, in NPOE was improved by a factor of from 1.7 to 10 or more as compared to that of the Tl-Por represented by the formula R-1. Of those, the Tl-Por represented by each of the formulae P-4 and the P-9 exhibited a solubility as high as 1% or more with respect to NPOE. (The solubility of the Tl-Por represented by the formula R-1 was 0.09%.)

### Evaluation of Uniformity of ISM

In each of Examples 1 to 7, the ISM was prepared through use of the substituent-introduced Tl-Por represented by each of the formulae P-1, P-4, P-7, P-9, P-21, P-22, and P-27. In Comparative Example, the ISM was prepared through use of the Tl-Por represented by the formula R-1.

No clear aggregation on backlight photography was recognized in any of the ISMs of Examples 1 to 4. Aggregation was present but was minute in the ISM prepared through use of the Tl-Por represented by each of the formulae P-21, P-22, and P-27 of Examples 5 to 7. In addition, no crystal deposition of the Tl-Por on the membrane surface was observed in any of the ISMs of Examples 1 to 5. In contrast, large aggregation was recognized in the ISM of Comparative Example. In addition, crystal deposition of the Tl-Por represented by the formula R-1 with blue-violet metallic luster was recognized on the membrane surface.

### Evaluation of Electromotive Force

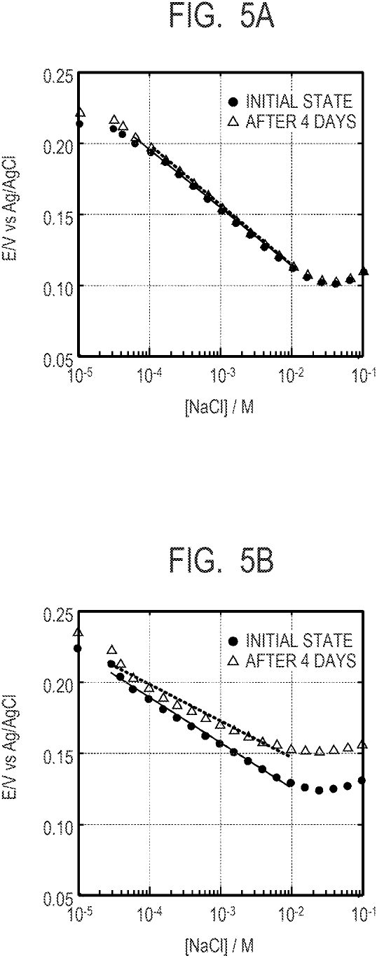

FIG. 5A and FIG. 5B are graphs for showing EMF responses when the concentration of NaCl in the aqueous solution is changed in the cases of using the ISEs of Example 4 and Comparative Example, respectively. In each of the figures, the plot indicated by the black circle markers represents a response in an initial state, and the plot indicated by the white triangle markers represents a response after storage in a NaCl aqueous solution at a concentration of 10 mM for 4 days from the initial measurement. The horizontal axis of the graph represents the concentration of NaCl (logarithm), and the vertical axis thereof represents an EMF. In each of the ISEs, the EMF is changed linearly with respect to the logarithm of the concentration of the target ion, and the slope thereof represents sensitivity to the target ion. In addition, in the case of an ISE for anions as in this embodiment, the slope thereof becomes a negative slope. In FIG. 5A, there are shown the characteristics of an ISE produced through use of the substituent-introduced Tl-Por represented by the formula P-9 (Example 4) by setting the concentration of NaTFPB to 50% of the optimum concentration. The initial slope value obtained therefrom was -41.1 mV dec⁻¹ (change (unit: mV) in EMF when the concentration was changed by an order of magnitude). In contrast, the initial slope shown in FIG. 5B of the ISE prepared through use of the Tl-Por represented by the formula R-1 of Comparative Example by setting the concentration of NaTFPB to 5.6% of the optimum concentration was -29.8 mV dec⁻¹. In Table 1, there is shown a list of the optimum concentration of NaTFPB of the ISE and the values of slopes at the optimum concentration in each of Examples 1 to 7 and Comparative Example. The optimum concentration of NaTFPB was determined from the magnitude of the absolute value of the initial slope.

As can be seen from the table, in each of Examples 1 to 7 using the substituent-introduced Tl-Pors represented by the formulae P-1, P-4, P-7, P-9, P-21, P-22, and P-27, the initial slope exhibited an absolute value of from -37.2 mV dec⁻¹ to -46.2 mV dec⁻¹, which was larger than that in Comparative Example.

### Evaluation of Stability

The NaCl concentration-dependent EMFs of each of the ISMs were measured before and after the ISM was immersed in a NaCl aqueous solution having a concentration of 10 mM and was left to stand for 4 days. Thus, the stability of the ISE using the Tl-Por was evaluated. The ISE using the Tl-Por represented by the formula P-9 (Example 4) shown in FIG. 5A exhibited a slope of -42.4 mV dec⁻¹ that remained almost unchanged from the initial slope even after 4 days. In contrast, in the ISE using the Tl-Por represented by the formula R-1 of Comparative Example shown in FIG. 5B, the sensitivity was decreased to -21.4 mV dec⁻¹ that was 0.72 times the initial sensitivity. In Table 1, there is shown a list of the slope of the ISE after immersion for 4 days and a change ratio of the slope (value obtained by dividing the slope after 4 days by the initial slope) of each of Examples 1 to 7 and Comparative Example. As can be seen from the table, in each of Examples 1 to 7 using the substituent-introduced Tl-Pors represented by the formulae P-1, P-4, P-7, P-9, P-21, P-22, and P-27, a change ratio of the slope exhibited a value of from 0.92 to 1.04, which was larger than the ratio (0.72) of the ISE using the Tl-Por represented by the formula R-1 of Comparative Example.

TABLE 1

List of EMF response of ISE when concentration of NaCl in aqueous solution is changed

| | Used Tl-Por | Initial slope mV dec⁻¹ | Slope after 4 days mV dec⁻¹ | Change ratio of slope | Optimum concentration of NaTFPB /% |
|---|---|---|---|---|---|
| Example 1 | P-1 | -43.0 | -43.8 | 1.02 | 20 |
| Example 2 | P-4 | -37.2 | -38.4 | 1.03 | 50 |
| Example 3 | P-7 | -41.4 | -42.9 | 1.04 | 50 |
| Example 4 | P-9 | -41.1 | -42.4 | 1.03 | 50 |
| Example 5 | P-21 | -46.2 | -42.7 | 0.92 | 5.6 |
| Example 6 | P-22 | -37.3 | -36.5 | 0.98 | 20 |
| Example 7 | P-27 | -41.5 | -41.2 | 0.99 | 5.6 |
| Comparative Example | R-1 | -29.8 | -21.4 | 0.72 | 5.6 |

### Discussion

It was found from the solubility evaluation and the uniformity evaluation that the solubility of each of the Tl-Pors in the membrane solvent and the uniformity thereof in the ISM matrix were improved by introducing a substituent into the Tl-Por. This is probably because the introduction of a relatively flexible functional group into a π-conjugated spreading porphyrin ring improved the affinity of the Tl-Por for the membrane solvent and the polymer that were relatively flexible molecules. In addition, it is probably because the suppression of association by steric hindrance through the introduction of the substituents functioned that the following two kinds of Tl-Pors each exhibited particularly high solubility: the Tl-Por represented by the formula P-4 having three substituents at the ortho- and para-positions of a phenyl group of tetraphenylporphyrin; and the Tl-Por represented by the formula P-9 having two substituents at the meta-positions thereof. As a result, the Tl-Por represented by the formula R-1 of Comparative Example, which was difficult to be stably present in the membrane solvent and the ISM matrix, and which was aggregated in the membrane and deposited on the membrane surface as a crystal, became able to be stably held in the membrane solvent and the ISM matrix.

It was found from the electromotive force evaluation that the ISE using the substituent-introduced Tl-Por of each of Examples 1 to 7 exhibited the slope having an absolute value larger than that of the slope of the ISE using the Tl-Por represented by the formula R-1 of Comparative Example. This is probably because the introduction of a substituent into the Tl-Por allowed the Tl-Por to be stably present in the matrix, to thereby increase the amount of the Tl-Por that effectively functioned as the ionophore. In contrast, it is conceived that the Tl-Por represented by the formula R-1 of Comparative Example was difficult to be stably present in the membrane solvent and the matrix, and was hence aggregated in the membrane and deposited on the membrane surface as a crystal, with the result that the amount of the Tl-Por that effectively functioned as the ionophore in the ISM was reduced, resulting in relatively low sensitivity.

It was found from the stability evaluation that the ISE using the ISM of each of Examples 1 to 7 using the substituent-introduced Tl-Por exhibited sensitivity that was more stable than that of the ISE using the ISM of Comparative Example. This is probably because that the introduction of a substituent into the Tl-Por stabilized the number of Tl-Por molecules that effectively functioned as the ionophore in the ISM, resulting in stable ISE characteristics. In contrast, in the case of the Tl-Por represented by the formula R-1 used in Comparative Example, it is conceived that the number of Tl-Por molecules that effectively functioned as the ionophore in the ISM was gradually reduced, resulting in decreased ISE characteristics.

The following was found from the above-mentioned results.

The introduction of a substituent into the Tl-Por improved the solubility in the membrane solvent, the uniformity in the ISM matrix, and the stability.

Through use of the ISM using the substituent-introduced Tl-Por, the sensitivity of each of the ISE and the ion sensor was improved.

Through use of the ISM using the substituent-introduced Tl-Por, the stability with time of an ion-selective potential response of each of the ISE and the ion sensor was improved.

Specifically, it was verified that according to this embodiment, an ISM containing a Tl-Por with improved stability as an ionophore, an ISE and an ion sensor each having improved sensitivity, and an ISE and an ion sensor each having improved potential response stability were able to be provided, and as a result, the value of a device using an ISE, which was typified by an ion sensor or a specimen testing device, was able to be improved.

According to this embodiment, an ISM containing a Tl-Por with improved stability as an ionophore can be provided.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2022-007483, filed Jan. 20, 2022, and Japanese Patent Application No. 2022-198727, filed Dec. 13, 2022, which are hereby incorporated by reference herein in their entirety.

What is claimed is:

1. An ion-selective membrane comprising:
a compound represented by the followingformula (1);
a polymer; and
a membrane solvent: 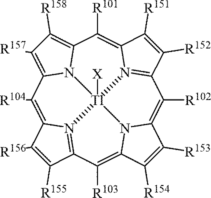
in the formula (1),
(i) R¹⁵¹ to R¹⁵⁸ and R¹⁰¹ to R¹⁰⁴ each independently represent a hydrogen atom, a saturated or unsaturated alkyl group having 1 or more and 20 or less carbon atoms that may have a substituent, an alkoxy group having 1 or more and 20 or less carbon atoms that may have a substituent, an alkylcarbonyl group having 1 or more and 20 or less carbon atoms that may have a substituent, an aryl group having 5 or more and 20 or less carbon atoms that may have a substituent, an aryloxy group having 5 or more and 20 or less carbon atoms that may have a substituent, an aralkyl group having 6 or more and 30 or less carbon atoms that may have a substituent, or a halogen atom,
(ii) the substituent of the alkyl group in the (i) is selected from the group consisting of: an alkoxy group having 1 or more and 4 or less carbon atoms; an alkylcarbonyl group having 1 or more and 4 or less carbon atoms; an alkyloxycarbonyl group having 1 or more and 4 or less carbon atoms; an alkylcarbonyloxy group having 1 or more and 4 or less carbon atoms; an aryl group having 5 or more and 20 or less carbon atoms; a cyano group; and a halogen atom,
(iii) the substituent of each of the alkoxy group, the alkylcarbonyl group, the aryl group, the aryloxy group, and the aralkyl group in the (i) is selected from the group consisting of: a saturated or unsaturated alkyl group having 1 or more and 20 or less carbon atoms; an alkoxy group having 1 or more and 20 or less carbon atoms; an alkylcarbonyl group having 1 or more and 20 or less carbon atoms; an alkyloxycarbonyl group having 1 or more and 20 or less carbon atoms; an alkylcarbonyloxy group having 1 or more and 20 or less carbon atoms; an aryl group having 5 or more and 20 or less carbon atoms; a cyano group; and a halogen atom,
(iv) at least one of the R¹⁵¹ to the R¹⁵⁸ and the R¹⁰¹ to the R¹⁰⁴ represents a group except a hydrogen atom, and
(v) X represents an anion.

2. The ion-selective membrane according to claim 1, wherein the compound represented by the formula (1) is represented by the following formula (2): 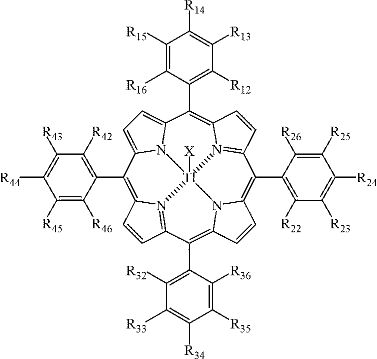
in the formula (2),
(i) R¹² to R¹⁶, R²² to R²⁶, R³² to R³⁶, and R⁴² to R⁴⁶ each independently represent a hydrogen atom, a saturated or unsaturated alkyl group having 1 or more and 20 or less carbon atoms that may have a substituent, an alkoxy group having 1 or more and 20 or less carbon atoms that may have a substituent, an alkylcarbonyl group having 1 or more and 20 or less carbon atoms that may have a substituent, an aryl group having 5 or more and 20 or less carbon atoms that may have a substituent, an aryloxy group having 5 or more and 20 or less carbon atoms that may have a substituent, an aralkyl group having 6 or more and 30 or less carbon atoms that may have a substituent, a hydroxy group, or a halogen atom,
(ii) the substituent of the alkyl group in the (i) is any one of an alkoxy group having 1 or more and 4 or less carbon atoms, an alkylcarbonyl group having 1 or more and 4 or less carbon atoms, an alkyloxycarbonyl group having 1 or more and 4 or less carbon atoms, an alkylcarbonyloxy group having 1 or more and 4 or less carbon atoms, an aryl group having 5 or more and 20 or less carbon atoms, a cyano group, or a halogen atom,
(iii) the substituent of each of the alkoxy group, the alkylcarbonyl group, the aryl group, the aryloxy group, and the aralkyl group in the (i) is any one of a saturated or unsaturated alkyl group having 1 or more and 4 or less carbon atoms, an alkoxy group having 1 or more and 4 or less carbon atoms, an alkylcarbonyl group having 1 or more and 4 or less carbon atoms, an alkyloxycarbonyl group having 1 or more and 4 or less carbon atoms, an alkylcarbonyloxy group having 1 or more and 4 or less carbon atoms, an aryl group having 5 or more and 20 or less carbon atoms, a cyano group, or a halogen atom,
(iv) at least one of the R¹² to the R¹⁶, the R²² to the R²⁶, the R³² to the R³⁶, and the R⁴² to the R⁴⁶ represents a group except a hydrogen atom, and
(v) X represents an anion.

3. The ion-selective membrane according to claim 2, wherein at least one of the R¹³, the R¹⁵, the R²³, the R²⁵, the R³³, the R³⁵, the R⁴³, or the R⁴⁵ in the formula (2) represents an alkoxy group having 1 or more and 20 or less carbon atoms that may have a substituent.

4. The ion-selective membrane according to claim 1, wherein the compound represented by the formula (1) is represented by the following formula (3): 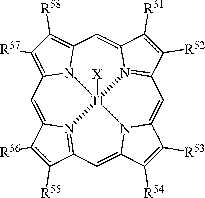
in the formula (3),
(i) R⁵¹ to R⁵⁸ each independently represent a hydrogen atom, a saturated or unsaturated alkyl group having 1 or more and 20 or less carbon atoms that may have a substituent, an alkoxy group having 1 or more and 20 or less carbon atoms that may have a substituent, an alkylcarbonyl group having 1 or more and 20 or less carbon atoms that may have a substituent, an aryl group having 5 or more and 20 or less carbon atoms that may have a substituent, an aryloxy group having 5 or more and 20 or less carbon atoms that may have a substituent, an aralkyl group having 6 or more and 30 or less carbon atoms that may have a substituent, or a halogen atom,
(ii) the substituent of the alkyl group in the (i) is any one of an alkoxy group having 1 or more and 4 or less carbon atoms, an alkylcarbonyl group having 1 or more and 4 or less carbon atoms, an alkyloxycarbonyl group having 1 or more and 4 or less carbon atoms, an alkylcarbonyloxy group having 1 or more and 4 or less carbon atoms, an aryl group having 5 or more and 20 or less carbon atoms, a cyano group, or a halogen atom,
(iii) the substituent of each of the alkoxy group, the alkylcarbonyl group, the aryl group, the aryloxy group, and the aralkyl group in the (i) is any one of a saturated or unsaturated alkyl group having 1 or more and 4 or less carbon atoms, an alkoxy group having 1 or more and 4 or less carbon atoms, an alkylcarbonyl group having 1 or more and 4 or less carbon atoms, an alkyloxycarbonyl group having 1 or more and 4 or less carbon atoms, an alkylcarbonyloxy group having 1 or more and 4 or less carbon atoms, an aryl group having 5 or more and 20 or less carbon atoms, a cyano group, or a halogen atom,
(iv) at least one of the R⁵¹ to the R⁵⁸ represents a group except a hydrogen atom, and
(v) X represents an anion.

5. The ion-selective membrane according to claim 1, wherein the ion-selective membrane is configured to select a halide ion.

6. The ion-selective membrane according to claim 1, wherein the ion-selective membrane is configured to select a chloride ion.

7. The ion-selective membrane according to claim 1, wherein the X in the formula (1) represents any one of a chloride ion, a bromide ion, an iodide ion, a fluoride ion, a trifluoroacetate ion, a tetrafluoroborate ion, a hexafluorophosphate ion, an acetate ion, a nitrate ion, a perchlorate ion, or a thiocyanate ion.

8. The ion-selective membrane according to claim 1, wherein the X in the formula (1) represents a chloride ion.

9. The ion-selective membrane according to claim 1, wherein the polymer contains at least one kind selected from a polymer of ethylene that may have a substituent, a polymer of styrene that may have a substituent, a polymer of an acrylic acid ester, a polymer of a methacrylic acid ester, a polymer of a diene-based compound, polyurethane, a polymer having a siloxane bond, and a cellulose derivative.

10. The ion-selective membrane according to claim 1, wherein the polymer contains at least one kind selected from polyvinyl chloride, polystyrene, polymethyl acrylate, polymethyl methacrylate, polyvinyl acetate, polybutadiene, polyisoprene, polyacrylonitrile, and cellulose acetate.

11. The ion-selective membrane according to claim 1, wherein the membrane solvent contains one of a phthalic acid ester or an ortho-nitrophenyl ether.

12. The ion-selective membrane according to claim 1, wherein the membrane solvent contains at least one kind selected from ortho-nitrophenyl octyl ether (NPOE), dioctyl phthalate, and 2-fluoro-2′-nitrophenyl ether.

13. The ion-selective membrane according to claim 1, further comprising an ionic additive.

14. The ion-selective membrane according to claim 13, wherein the ionic additive contains a hydrophilic cation and a hydrophobic anion.

15. An ion-selective electrode comprising:
an electrode including at least one kind of conductor; and
the ion-selective membrane of claim 1.

16. An ion sensor comprising:
the ion-selective electrode of claim 15;
a reference electrode; and
a measuring instrument configured to measure a potential difference between the ion-selective electrode and the reference electrode.

17. A specimen testing device comprising:
the ion-selective electrode of claim 15; and
a specimen supply mechanism configured to supply a specimen to the ion-selective membrane.

18. A complex compound represented by the following formula (4): 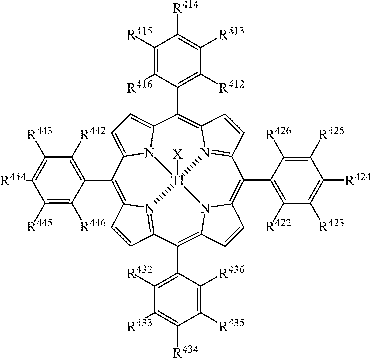
in the formula (4),
(i) R⁴¹² to R⁴¹⁶, R⁴²² to R⁴²⁶, R⁴³² to R⁴³⁶, and R⁴⁴² to R⁴⁴⁶ each independently represent a hydrogen atom, a saturated or unsaturated alkyl group having 1 or more and 20 or less carbon atoms that may have a substituent, an alkoxy group having 1 or more and 20 or less carbon atoms that may have a substituent, an alkylcarbonyl group having 1 or more and 20 or less carbon atoms that may have a substituent, an aryl group having 5 or more and 20 or less carbon atoms that may have a substituent, an aryloxy group having 5 or more and 20 or less carbon atoms that may have a substituent, an aralkyl group having 6 or more and 30 or less carbon atoms that may have a substituent, a hydroxy group, or a halogen atom,
(ii) the substituent of the alkyl group in the (i) is any one of an alkoxy group having 1 or more and 4 or less carbon atoms, an alkylcarbonyl group having 1 or more and 4 or less carbon atoms, an alkyloxycarbonyl group having 1 or more and 4 or less carbon atoms, an alkylcarbonyloxy group having 1 or more and 4 or less carbon atoms, an aryl group having 5 or more and 20 or less carbon atoms, a cyano group, or a halogen atom,
(iii) the substituent of each of the alkoxy group, the alkylcarbonyl group, the aryl group, the aryloxy group, and the aralkyl group in the (i) is any one of a saturated or unsaturated alkyl group having 1 or more and 4 or less carbon atoms, an alkoxy group having 1 or more and 4 or less carbon atoms, an alkylcarbonyl group having 1 or more and 4 or less carbon atoms, an alkyloxycarbonyl group having 1 or more and 4 or less carbon atoms, an alkylcarbonyloxy group having 1 or more and 4 or less carbon atoms, an aryl group having 5 or more and 20 or less carbon atoms, a cyano group, or a halogen atom,
(iv) at least one of the R⁴¹² to the R⁴¹⁶, the R⁴²² to the R⁴²⁶, the R⁴³² to the R⁴³⁶, and the R⁴⁴² to the R⁴⁴⁶ represents a group except a hydrogen atom,
(v) X represents an anion, and
(vi) at least one of the R⁴¹³, the R⁴¹⁵, the R⁴²³, the R⁴²⁵, the R⁴³³, the R⁴³⁵, the R⁴⁴³, or the R⁴⁴⁵ represents an alkoxy group having 1 or more and 20 or less carbon atoms that may have a substituent.

19. The complex compound according to claim 18, wherein, in the formula (4), the R⁴¹³, the R⁴¹⁵, the R⁴²³, the R⁴²⁵, the R⁴³³, the R⁴³⁵, the R⁴⁴³, and the R⁴⁴⁵ each represent an alkoxy group having 1 or more and 20 or less carbon atoms that may have a substituent.

20. The complex compound according to claim 18, wherein the X in the formula (4) represents any one of a halide ion, a trifluoroacetate ion, a tetrafluoroborate ion, a hexafluorophosphate ion, an acetate ion, a nitrate ion, a perchlorate ion, or a thiocyanate ion.

21. The complex compound according to claim 18, wherein the X in the formula (4) represents a chloride ion.

22. A complex compound represented by the following formula (5): 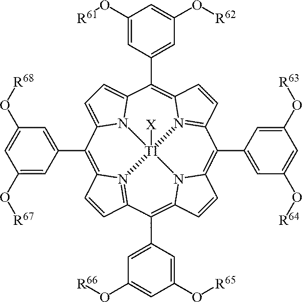
in the formula (5),
R⁶¹ to R⁶⁸ each independently represent a saturated or unsaturated alkyl group having 1 or more and 20 or less carbon atoms that may have a substituent,
the substituent is selected from the group consisting of: an alkoxy group having 1 or more and 4 or less carbon atoms; an alkylcarbonyl group having 1 or more and 4 or less carbon atoms; an alkyloxycarbonyl group having 1 or more and 4 or less carbon atoms; an alkylcarbonyloxy group having 1 or more and 4 or less carbon atoms; an aryl group having 5 or more and 20 or less carbon atoms; a cyano group; and a halogen atom, and
X represents an anion.

23. The complex compound according to claim 22, wherein the X in the formula (5) represents any one of a halide ion, a trifluoroacetate ion, a tetrafluoroborate ion, a hexafluorophosphate ion, an acetate ion, a nitrate ion, a perchlorate ion, or a thiocyanate ion.

24. The complex compound according to claim 22, wherein the X in the formula (5) represents a chloride ion.
